(19) United States
(12) Patent Application Publication
(10) Pub. No.: US 2015/0239941 A1
(43) Pub. Date: Aug. 27, 2015

(54) METHOD FOR EXTRACTING AND STABILISING PHYCOCYANIN AND THE USES THEREOF

(71) Applicant: ECOSYSTEM, Patary (FR)

(72) Inventor: Frederic Pottecher, Terminiers (FR)

(21) Appl. No.: 14/429,475

(22) PCT Filed: Sep. 13, 2013
(86) PCT No.: PCT/IB2013/058533

§ 371 Date: Mar. 19, 2015

(30) Foreign Application Priority Data
Sep. 20, 2012 (FR) 1258824

(51) Int. Cl. C07K 14/195; A61K 35/748; A61K 8/99

(52) U.S. Cl. CPC C07K 14/195; A61K 8/99; A61K 35/748

(57) ABSTRACT

Disclosed is a method for extracting and stabilizing phycocyanin from at least one material containing same. The method includes a step of macerating the material or materials in glycerol or a water/glycerol mixture.

The object of the invention is to provide a process for extracting and stabilizing phycocyanin and its applications in particular in diagnostics and in the medical, food or cosmetics field, or as food supplements.

Phycocyanin is a water soluble protein which is one of the main pigments of cyanobacteria as for example spirulin (Arthrospira platensis or Spirulina maxima) or AFA (Aphanizomenon Flos-Aquae). At rest, phycocyanin has a blue colour and a red fluorescence. It has a maximum absorption at 620 nm and an emission radiation at 635 nm. This quality makes it a natural fluorescent product which is a favoured label in biomedical diagnostics.

At the physiological level, phycocyanin is also an important antioxidant in particular to protect plasma proteins against oxidative modifications. The chemical structure of phycocyanin has a very close structural similarity with bilirubin (which is a haemoglobin degradation product) and quite similar properties.

Phycocyanin is a molecule of great interest in particular because of its beneficial properties for human and animal health. Phycocyanin is consumed in particular for its antioxidant properties and also for its ability to promote the production of stem cells.

However, it is an expensive molecule difficult to extract. It is further a molecule which is very quickly degraded, after extraction, in case of bacterial contamination, which raises the costs because in liquid form, in water, this molecule has to be extracted under sterile conditions and packaged in single doses.

Different extraction methods have been proposed, among which the tangential flow filtration, solvent accelerated extraction, cell lysis and two-phase aqueous extraction.

Patent FR 2 789 399 describes a 0.2 micrometre tangential flow filtration process which enables phycocyanin to be cold extracted in aqueous media and in a sterile way. However, this process demands high capital costs, because the tangential flow filtration materials are very expensive, and it involves packaging in single doses, for example glass ampoules, because after opening, the medium is no longer sterile and phycocyanin does not preserve in water under these conditions.

This process is very complicated to make with glycerol as a solvent in substitution for water, since glycerol has too high a viscosity to allow a tangential flow filtration to be made under good conditions.

The solvent accelerated extraction process which lasts less than thirty minutes is characterised by high temperature and pressure which maintain the solvent below its boiling point. In this method, several solvents have been tested: hexane, petroleum ether, ethanol and water, at temperatures of 60, 115 and 170° C. and different extractions times of 3.9 and 15 min. All these solvents raise problems: hexane and petroleum ether are toxic. Ethanol allows the most efficient extraction, but raises many problems: toxicity, prohibition in some religions and a much higher antioxidant activity loss than with hexane or petroleum ether which have very low extraction efficiencies of only 4.30% for hexane and 3.60% for petroleum ether. Water in turn raises preservation problems because in case of bacterial contamination, phycocyanin is degraded in a few days.

The most commonly employed cell lysis technique consists in alternating freeze-thaw phases followed by:

either an ultrasound treatment which breaks up cell membranes and thus facilitates the access to molecules present in the cytoplasm and intracellular organites;

or adding phosphate buffer solution pH 6.8 during the freeze-thaw step and then passing through a mixer, followed by a hydrochloric acid extraction step.

The separation and concentration of the different components are then carried out by a centrifugation step enabling the supernatant wherein phycocyanin is to be collected.

This technique has drawbacks given that phycocyanin in aqueous form is quickly degraded in case of bacterial contamination and thus has to be preserved in a sterile form or in the cold (freezing), which causes high additional costs.

The two-phase aqueous extraction involves the separation of molecules between two or more immiscible aqueous phases. Polyethylene glycol (PEG) and potassium phosphate are added to a given amount of fresh phycocyanin extract. After mixing and settling, the system is separated into two phases. These phases are then analysed by ion exchange chromatography and UV spectroscopy to assess purity and amount of phycocyanin and total proteins. The PEG and potassium phosphate mixture is used to have a partition coefficient between phycocyanin and other proteins. This system is then subjected to the two-phase aqueous extraction. To increase purity, an ion exchange chromatography is carried out. If this extraction method enables a high phycocyanin concentration to be obtained, it has however the drawback of requiring numerous and complex steps and the usage of sophisticated expensive equipment.

There is thus a need for a process for obtaining phycocyanin which is easily implemented and economically satisfactory.

A further requirement is on stability of the phycocyanin solution obtained. Indeed, it is known that in aqueous media, heat or ultraviolet sterilization degrades the phycocyanin molecule and losses its entire efficiency after opening the package, implying a single dose package. Chemical preservatives have a toxicity which makes them incompatible for a food usage. Only tests with essential oils have given acceptable results. Ravintsara (Cinnamomum Camphora) essential oil delays the bacterial growth while remaining compatible with a food usage, but this treatment is only suitable for a short term preservation of phycocyanin because bacterial growths are slowed down but not annihilated. It is not possible either to strongly acidify the medium to prevent bacteria growth because phycocyanin is very sensitive to pH and any significant variation causes a precipitation of the molecules. Phycocyanin becomes unstable and precipitates below pH 5 and beyond pH 7.5

Freezing is a solution simple to implement, but it makes transport very complicated and very expensive because of the necessity to maintain the cold chain.

Alcohol (ethanol) with a high concentration enables phycocyanin to be stabilized, but affects the properties of phycocyanin which loses most of its antioxidant properties. In addition, alcohol is problematic because of multiple religious prohibitions to which it is subjected. Further, its use faces numerous technical problems: it is a very flammable product, having a dependency (alcohol toxicity), to which commercial constraints are applied, since alcohol products are subjected to numerous taxes and prohibitions.

Other solvents or chemical products used for extraction processes: hexane, petroleum ether, phosphate buffer solution, polyethylene glycol are expensive and have significant food toxicity problems.

Solutions found by the industries to allow a long term preservation of phycocyanin at room temperature are the following ones:
either phycocyanin is, after extraction and concentration, preserved in the form of a dehydrated dry powder which dramatically decreases its efficiency because drying affects molecules as well as their three-dimensional structure and results in losing or decreasing biological and fluorescence properties of the molecule;
or phycocyanin is preserved in a liquid form in aqueous media, which implies expensive and complex extraction, clarification and sterile filtration processes followed by packaging in single doses (for example glass ampoules), because after opening, the medium is contaminated with bacteria and the consumption has therefore to be immediate.

It is known that the liquid form is to be favoured, since phycocyanin in this form is much more bioavailable than in the dry form because it remains in its native form and preserves its fluorescence properties.

These processes dramatically rise prices and make active and highly purified phycocyanin very expensive. Its price can reach US$50 per milligram purified.

Phycocyanin production at a reasonable cost without degrading its properties is thus still challenging.

Researches made by the inventor have shown that these problems could be overcome satisfactorily by carrying out the extraction step with a new solvent.

One purpose of the invention is thus to provide a new process for extracting and stabilizing phycocyanin.

It also aims at providing phycocyanin solutions or extracts having optionally other nutrients, with a great stability, advantageously usable in multi-dose packages for applications in particular in diagnostics and in medical, food or cosmetics fields, or as food supplements.

The process for extracting phycocyanin from at least one material containing it is characterised in that it comprises a step of macerating the material(s) in glycerol or a water/glycerol mixture.

The use of a water/glycerol mixture is of interest regarding its lower cost, quicker filtration, the product being more fluid and allows an extraction with a better efficiency. The phycocyanin content is higher in the filtrate when a water/glycerol mixture is used instead of pure glycerol.

Advantageously, the water/glycerol ratio can range up to 60/40.

Glycerol is a molecule which has many advantages. Glycerol is an approved food ingredient (E422) which is recognized as very safe and which is consequently allowed quantum satis. Glycerol is halal, Kosher and consumable by vegetarians and vegans. Finally, glycerol has the advantage of having a pleasant sweet taste and be low calorie. In addition, this is a molecule which is naturally produced by living organisms. When the body uses fats stored as an energy source, glycerol and fatty acids are released in blood.

Further, glycerol is an excellent natural preservative because it stabilizes proteins and prevents their aggregation as well as bacteria growth. Glycerol is also a cryoprotectant which prevents ice crystals from being formed.

Preferably, the material(s) containing phycocyanin used in the process of the invention are in a dry or fresh powder form.

When the extraction is sufficient, for example after 2 weeks of maceration at room temperature, a filtration step is carried out which results in obtaining a filtrate containing the extracted phycocyanin and a retentate.

Said material(s) is (are) advantageously selected from spirulin (Arthrospira platensis or Spirulina maxima) and/or AFA (Aphanizomenon Flos-Aquae).

Surprisingly, phycocyanin is naturally extracted and gradually concentrated in glycerol over time.

Advantageously, the filtrate obtained also contains micro-nutrients which are naturally extracted from the material(s) used.

According to a further arrangement of the invention, glycerol is heated to increase the extraction rate. Suitable temperatures are lower than 100° C., more specially than 70° C., preferably than 40° C. and are used more specially over a short period of time, to avoid degrading phycocyanin and to preserve fluorescence properties. The heated glycerol extracts phycocyanin much more quickly and the maceration duration can thus be shortened.

In a preferred embodiment of the process of the invention, the filtration is carried out in a frontal way, advantageously on a food-grade filter, for example of polyamide, in particular of nylon, with a fineness of 2 to 50 microns, preferably lower than 25 microns. This arrangements enables a clarified filtrate to be obtained wherein phycocyanin is active and is preserved with all its biological and fluorescence properties for a duration longer than one year. Phycocyanin is protected from bacteria and oxidation by glycerol. Up to 60% water in the extraction mixture, the risk of bacterial contamination or degradation of phycocyanin is very limited and biological properties are preserved over periods that can range up to several years. It is to be noted that the phycocyanin thus obtained preserves its fluorescence properties, which is an indirect proof of the preservation of its properties by the molecule and its non-denaturation.

In another preferred embodiment of the process of the invention as defined in one, several or all the above arrangements, the filtrate obtained is supplemented with pressurized CO₂, which enables a better preservation and provides a pleasant taste to the product.

Generally, the production cost of the phycocyanin obtained according to the invention is much lower than with usual processes because a simple cheap technology is implemented at room temperature. Furthermore, it is not necessary to have sterile conditions since glycerol protects phycocyanin from bacterial contaminations. Consequently, the product obtained is stable over time and it is therefore not necessary to package it into single dose ampoules.

In addition, after opening, the vial does not risk a bacterial contamination, which allows packaging in multi-dose packages, for example in 500 ml vials, corresponding to 30 daily doses for a human being. These packages are much more economical than a package in single dose ampoules.

It is to be noted that the filtration times which are long with a frontal filtration are not an issue since phycocyanin is protected from bacteria and oxidation throughout the process.

The materials used in the extraction step generally comprise numerous nutrients of interest. Thus, spirulin contains, in a perfectly assimilable form, numerous micro-nutrients. Among the elements reputed to be the most efficient, there are: Vitamins A, B2, B8, B12, PP, D, Superoxide dismutase, selenium, magnesium, zinc.

Most of the micro-nutrients of the materials used can be found in the liquid extract obtained according to the process of the invention and they also benefit from the protecting effect of glycerol.

The invention is also directed to the filtrates/extracts obtained. These filtrates/extracts comprise a solution in glycerol or a water/glycerol mixture of phycocyanin with one or more of the nutrients present in the material(s) used for the extraction.

In an alternative, the filtrates are added with pressurized CO₂.

In view of the above discussed advantages exhibited by these filtrates/extracts, their applications in the food, medical or cosmetics field, including toothpastes, or even as food supplements, prove to be particularly interesting. The filtrates/extracts of the invention can thus be used, in particular, as ingredients in the manufacture of cosmetics such as beauty creams with in particular an anti-wrinkle action or even in the preparation of toothpastes with a beneficial effect against gum bleeding.

There will be also mentioned the use of the filtrates/extracts of the invention in substitution of glycerol usually used in cosmetic preparations.

According to another aspect, the invention is directed to the packages of said extracts, in particular in multi-dose form. These packages are particularly suitable for their various applications.

The invention is also directed to new products, to the retentates obtained at the end of the filtration step. These retentates are also usable in the food, medical or cosmetics field or as food supplements.

Further characteristics and advantages of the invention are given in the following example by way of illustrating purposes.

### Example

800 grams of spirulin powder, AFA, or a mixture of both are mixed in 10kg of glycerol. They are left to macerate during 2 weeks at room temperature. A slow frontal filtration is then carried out, with a compatible food-grade filter, (for example nylon filter) having a fineness of 25 microns, given that the filter fineness has to be between 2 and 50 microns).

At the end of the filtration, about 7 litres of filtrate and 3 litres of retentate are obtained.

The filtrate which contains phycocyanin is packaged in multi-dose vials, for example in a 500 ml vial which corresponds to a sufficient quantity for about 30 days of use for a human being. Such a vial can be preserved without any problem for 3 years at room temperature.

Analyses performed by spectrophotometry have shown that this process enables phycocyanin concentrations exceeding 900 mg per litre of filtrate to be commonly obtained.

In addition, the retentate is readily recoverable on the filter and can be upgraded as food in view of its protein richness. The retentate can be used, for example, to enrich rations intended to animal feeding.

1. A process for extracting and stabilizing phycocyanin from at least one material containing it, the method comprising a step of macerating the at least one material in glycerol or a water/glycerol mixture.

2. The process according to claim 1, wherein the water/glycerol ratio is at most 60/40.

3. The process according to claim 1, wherein the material(s) used is (are) in the form of a dry or fresh powder.

4. The process according to claim 1, wherein at the end of the step of extraction by maceration, the material(s) used is (are) separated by filtering the glycerol and phycocyanin mixture, which results in a filtrate containing the phycocyanin and a retentate.

5. The process according to claim 1, wherein said materials are selected from spirulin (Arthrospira platensis or Spirulina maxima) and/or AFA (Aphanizomenon Flos-Aquae).

6. The process according to claim 1, wherein glycerol is heated at a temperature not exceeding 70° C., preferably lower than 40° C.

7. The process according to claim 4, wherein the filtration is carried out in a frontal way.

8. The process according to claim 4, wherein the filtration is carried out with a food-grade filter, for example of polyamide, in particular of nylon, with a fineness of 2 to 50 microns, preferably lower than 25 microns.

9. The process according to claim 1, wherein the method further includes adding pressurized carbon dioxide to the filtrate obtained.

10. Products as obtained in the process according to claim 1, containing the extracts containing in a glycerol or water/glycerol solution phycocyanin and one or more of the nutrients present in the material(s) used for the extraction.

11. The products according to claim 10, being packaged as single or multi-doses.

12. The products according to claim 10, for use as diagnostic agents, as drugs or in the food or cosmetics field, or as food supplements.

13. The process according to claim 2, wherein the material(s) used is (are) in the form of a dry or fresh powder.

14. The process according to claim 2, wherein at the end of the step of extraction by maceration, the material(s) used is (are) separated by filtering the glycerol and phycocyanin mixture, which results in a filtrate containing the phycocyanin and a retentate.

15. The process according to claim 3, wherein at the end of the step of extraction by maceration, the material(s) used is (are) separated by filtering the glycerol and phycocyanin mixture, which results in a filtrate containing the phycocyanin and a retentate.

16. The process according to claim 2, wherein said materials are selected from spirulin (Arthrospira platensis or Spirulina maxima) and/or AFA (Aphanizomenon Flos-Aquae).

17. The process according to claim 3, wherein said materials are selected from spirulin (Arthrospira platensis or Spirulina maxima) and/or AFA (Aphanizomenon Flos-Aquae).

18. The process according to claim 4, wherein said materials are selected from spirulin (Arthrospira platensis or Spirulina maxima) and/or AFA (Aphanizomenon Flos-Aquae).

19. The process according to claim 2, wherein glycerol is heated at a temperature not exceeding 70° C., preferably lower than 40° C.

20. The process according to claim 3, wherein glycerol is heated at a temperature not exceeding 70° C., preferably lower than 40° C.
